(19) United States
(12) Patent Application Publication
(10) Pub. No.: US 2020/0229717 A1
(43) Pub. Date: Jul. 23, 2020

(54) METHOD FOR OPERATING A BLOOD PRESSURE MEASURING DEVICE AND ARRANGEMENT FOR MEASURING THE PRESSURE IN A BLOOD VESSEL

(71) Applicant: REDWAVE MEDICAL GMBH, Jena (DE)

(72) Inventors: Verena Dittrich, Leipzig (DE); Chris Stockmann, Jena (DE); Andreas Mainka, Jena (DE)

(21) Appl. No.: 16/632,138

(22) PCT Filed: Jul. 31, 2018
(86) PCT No.: PCT/EP2018/070701

§ 371 Date: Jan. 17, 2020

(30) Foreign Application Priority Data
Jul. 31, 2017 (DE) 10 2017 117 337.4

(51) Int. Cl. A61B 5/021; A61B 5/022; A61B 5/00; A61B 5/025

(52) U.S. Cl. CPC A61B 5/02116; A61B 5/02233; A61B 5/025; A61B 5/7225; A61B 5/7257; A61B 5/7264

(57) ABSTRACT

The invention relates to a method for operating a blood pressure measuring device, comprising the method steps: performing a blood pressure measurement using the blood pressure measuring device, consisting of a cuff for placing around an extremity, a pump for inflating the sleeve and a pressure sensor for registering the cuff pressure, inflation of the cuff taking place in an inflation phase and release of the pressure in the sleeve taking place in a release phase, registering and storing the pressure profile in the cuff over time during the inflation phase and/or during the release phase in a control and storage unit, extracting a pulse-like signal component from the pressure profile over time of the measured cuff pressure during the inflation phase and/or the release phase by a signal analysis inside the control and storage unit, signal analysis of the extracted in pulse-like signal component.

The invention relates to a method for operating a blood pressure measuring device according to claim 1 and an arrangement for measuring the pressure in a blood vessel according to claim 9.

Oscillometrically operating blood pressure measuring devices are known. These are usually operated by first placing a cuff around one limb, e.g. the upper arm. The cuff is inflated by a pump or by hand. The pressure of the cuff interrupts the blood flow in a vessel running inside the limb. The pressure in the cuff is then released so that the blood flow in the squeezed vessel can be detected again. The blood pressure in this blood vessel results from the pressure currently prevailing in the cuff. Such a method can be used to determine, for example, the mean arterial pressure and from this both the systolic and diastolic blood pressure in the blood vessel.

The systolic and diastolic blood pressure are essential parameters with which the circulatory state of a test person can be characterized. However, in many cases these parameters are not sufficient. Very often, truly detailed statements about the hemodynamics of a living being can only be made if a more precise knowledge of the vascular properties is available. However, these cannot be obtained from a simple blood pressure measurement of the known kind, but require an analysis of the pulse waves.

The patent US 2017/0181648 A1 discloses a method which is able to determine blood pressure values in the upper arm very robustly. Two special pulse wave measuring units are used at the upper and lower end of the cuff and the pulse wave signals are offset against each other. However, as with any other standard blood pressure monitor, it is not possible to determine the vascular properties and hemodynamics of a living being.

Pulse wave analysis, on the other hand, uses central blood pressure and pulse wave velocity (PWV) to provide information about the stiffness of the arterial vessel wall and provides statements that go far beyond those that can be derived from blood pressure values measured on the upper arm, as used in home measurements, for example. Central blood pressure and PWV are independent predictors of cardiovascular events (myocardial infarction, stroke, death) and are a promising way to differentiate risk patients, especially those with intermediate cardiovascular risk.

In the majority of studies, the central blood pressure measured in the aorta has a much higher predictive value than the peripheral blood pressure measured in the upper arm. In addition, more recent data in the form of pilot studies indicate that both hypertension and heart failure therapy are better if they are based on central blood pressure and not on (conventional) upper arm blood pressure.

However, systems known from the prior art, which support pulse wave analysis, are expensive and complex to use and therefore especially unsuitable for the untrained layman, but also for broad practical application. In addition, conventional pulse wave analysis requires a modified and extended measurement procedure that cannot be realized in a controlled manner, especially under home conditions.

In accordance with the usual procedures for this purpose, a special target pressure, i.e. a pressure level, is approached and held for several seconds, usually 10 seconds. Such a measurement method is not only unpleasant for the patient, but the additional measurement effort also increases the susceptibility to errors, for example due to movement artefacts. These methodological requirements place special demands on the equipment used for measurement. Special devices must be developed, which are complex and associated with considerable costs. The algorithms used for the measurement are only compatible with the respective hardware and cannot be used on other platforms.

Patent DE 10 2004 011 779 B4 discloses, among other things, an arrangement which describes a standard blood pressure measuring device. This is used in this or a similar way, especially to compare the waveform of the individual pulse waves with each other and thus to reliably detect disturbances and artefacts. Such an arrangement is inexpensive, robust and therefore widespread worldwide and is used in the majority of standard sphygmomanometers.

Another very big problem is that when carrying out a pulse wave analysis, only a single transfer function is used for the whole population, which results in a significant uncertainty and loss of quality. In addition, this technology offers little information about other hemodynamic parameters, since only the pulse waves at the constant pressure level are evaluated.

Therefore, it is the object of the invention to specify a method with which the properties of aortal pulse waves can be determined with a minimum of effort, wherein the method sought should allow a complete pulse wave analysis with additional hemodynamic parameters. The aim is to simplify, robustly design and extend the data basis of pulse wave analysis in such a way that pulse wave analysis can be made available to the broad mass of physicians in private practice and, in particular, to patients at home in a simple and cost-effective manner.

The solution of the problem is achieved by a method for operating a blood pressure measuring device with the features of claim 1 and an arrangement for measuring the pressure in a blood vessel with the features of claim 9. The subclaims contain expedient or advantageous embodiments of the method or device.

According to the invention, the method is carried out with the following method steps:

The first step is to perform a blood pressure measurement with the blood pressure measuring device, consisting of a cuff to be wrapped around a limb, a pump to inflate the cuff and a pressure sensor to register the pressure inside the cuff. The cuff is inflated in an inflation phase and the pressure in the cuff is released in a release phase.

During the inflation phase and/or during the release phase, the temporal pressure curve in the cuff is registered and stored in a control and storage unit. A pulse-like signal component is then extracted from the temporal pressure curve of the measured cuff pressure during the inflation phase and/or the release phase by a signal analysis within the control and storage unit in conjunction with a signal analysis of the extracted pulse-like signal component.

The signal analysis takes place in the control and storage unit with the following steps:

In a first step, the pulse-like signal component is broken down into data about individual pulse waves. The data of the respective individual pulse waves are then combined from values of at least one calculation function from a given calculation function set, which may also be flexibly and/or autonomously expandable, to generate a respective classified pulse wave.

Then the respective classified single pulse wave is transformed. The transformation into a pulse-wave-specific amplitude and phase spectrum for each classified single period has proven to be suitable, but is not limited to it. Finally, the transformed data is evaluated to determine hemodynamic parameters.

In principle, the method according to the invention is initially based on the known method of ordinary blood pressure measurement. However, in contrast to conventional blood pressure measurement, in which an arterial blood flow is stopped via the cuff and is detected again when the pressure is released from the cuff in a dosed manner, the pressure in the cuff is registered in its time course during the entire inflation phase and/or the entire release phase, according to the invention. A pulse-like component is extracted from this recorded time course in one or both phases. After further signal analysis, this pulse-like component measured on the arm or another extremity first provides information about individual pulse waves from the arm or another extremity and finally about the respective aortal pulse waves of the test person. In contrast to the otherwise known measuring methods for aortal pulse waves, the usual procedure for blood pressure measurement is sufficient according to the invention to detect and analyze the aortal and thus the prevailing pulse wave generated and prevailing inside the body with a minimum of equipment and with minimized stress for the test person.

A further aspect in accordance with the invention is a more complex signal analysis of the pulse-like signal component, which is linked to a shape analysis of each individual period in the entire pulse-like signal component. This provides information on a list of individual periods, which allows a deeper insight into the data volume of the pulse-like signal component. In addition, each individual pulse, i.e. the signal course in each individual period, is also classified in its form. This is done by combining the signal waveform of each individual period from predefined calculation functions that can also be expanded optionally in a flexible and/or autonomous manner. In contrast to the conventional detection and analysis of aortal pulse waves, a transformation is thus not only performed by means of one calculation function, but several calculation functions. Details of the signal course are simultaneously determined, objectively classified and, after a transformation into, for example, the period-specific amplitude and phase spectrum, its properties are output as characteristics.

In a first embodiment, the pulse-like signal component is broken down into data over individual periods. A basepoint detection in the temporal course of the pulse-like signal component has proven to be suitable, but is not limited to it. This allows actual period trends and limits of individual periods to be reliably identified.

In one embodiment of the method, at least two weighted calculation functions are combined when combining the data of the respective individual periods to generate the respective classified individual period. This makes it possible not only to assign signal characteristics of individual periods to one calculation function, but also to have intermediate assignments to several calculation functions carried out.

The transformation of the respective classified individual period is carried out in an embodiment of the method by means of a Fourier analysis unit into the period-specific amplitude and phase spectrum. The Fourier analysis unit can also be implemented in the form of an evaluation program.

In one embodiment of the method, a re-transformation of the period-specific amplitude and phase spectrum of the classified individual pulse wave is performed to determine the time course of the aortic pulse wave. The retransformation provides the aortic pulse curve of this specific class and results on hemodynamic parameters of pulse wave analysis.

In one embodiment, the registration of the temporal pressure curve in the inflation phase and/or in the release phase is carried out digitally with a fixed device-specific sampling rate and a fixed device-specific resolution, wherein subsequent sampling to a platform-independent sampling rate is carried out in conjunction with an approximation of the measured time curve of the cuff pressure. This makes it possible to evaluate and compare the pressure curves obtained independently of the platform and thus independent of the device.

In one embodiment of the method, a non-oscillating cuff pressure is separated out when the pulse-like signal component is extracted via a bandpass filter. Although the non-oscillating cuff pressure does not provide any information about the pulse wave as such, it does allow conclusions to be drawn about possible undesirable and falsifying influences on the measuring process.

In one embodiment of the method, the separated non-oscillating cuff pressure is checked for conformity with a standard curve, wherein an artefact parameter for indicating a measured value-distorting influence is determined from the degree of deviation of the non-oscillating pump trend from the linear curve. The artefact parameter is a measure of the quality of the signals and carries information about the examination. It describes the falsifying influence to which the entire process of pressure measurement has been subjected and how valid the calculated results are.

An arrangement for measuring the pressure in a blood vessel comprises a cuff for application around an extremity, a pump for inflating the cuff, a pressure sensor for registering the temporal pressure curve in the cuff and a control and storage unit for operating the pump and the pressure sensor and for storing the temporal pressure curve. The control and storage unit has a control and evaluation program for separating pulse-like components in the temporal pressure curve during an inflation and/or release phase of the temporal pressure curve and for a signal analysis of the separated pulse-like components.

The control and evaluation program can be uploaded and updated from an external storage medium via an interface to the control and storage unit in one embodiment of the device. The control and storage unit can thus be easily reconfigured in its operating mode.

In a further embodiment, the control and storage unit has an interface for reading out stored data, in particular the measured temporal pressure curve, to an external evaluation unit and/or a remote host. This enables external, remote and centralized evaluation as well as remote monitoring.

In one embodiment, the control and storage unit has a display for outputting parameters obtained from the pulse-like components of the pressure curve, in particular for outputting a heart rate and/or hemodynamic parameters from pulse wave analysis and/or a curve of an aortic pulse wave.

In a further embodiment, the control and storage unit has a switch-over facility between a first operating mode for carrying out a standard blood pressure measurement and a second operating mode for carrying out a measurement of the pulse-like components of the temporal pressure curve and for determining parameters of an aortal pulse wave. The blood pressure measuring device can therefore be operated in different operating modes.

The above-mentioned method and the device are used to determine and analyze at least one aortic pulse wave and to determine hemodynamic parameters of pulse wave analysis, such as aortic blood pressure.

The method and the arrangement will be explained in more detail in the following on the basis of embodiment examples. The enclosed figures serve to clarify this, wherein:

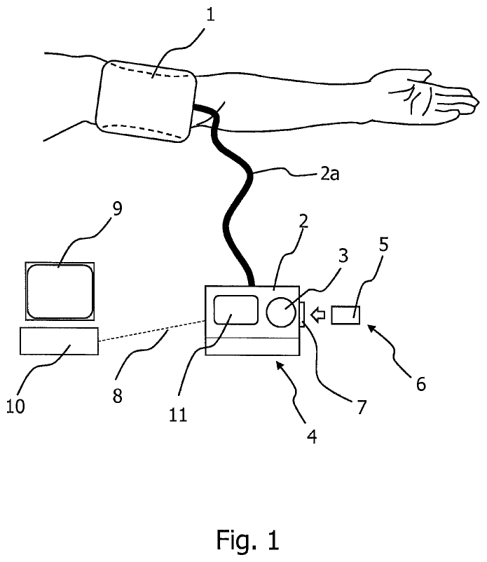

FIG. 1 shows a basic configuration of an arrangement for carrying out the method,

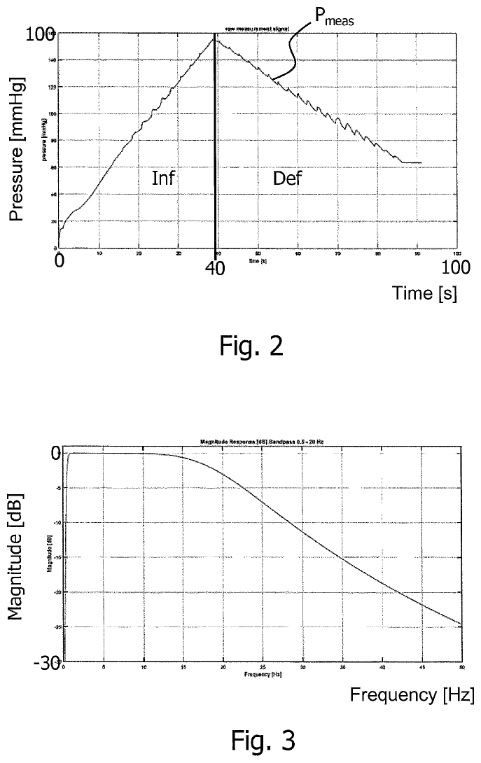

FIG. 2 shows an exemplary progression of the pressure within the cuff with an inflation and a release phase,

FIG. 3 shows an exemplary representation of the amplitude spectrum of the impulse response for an exemplary bandpass filter,

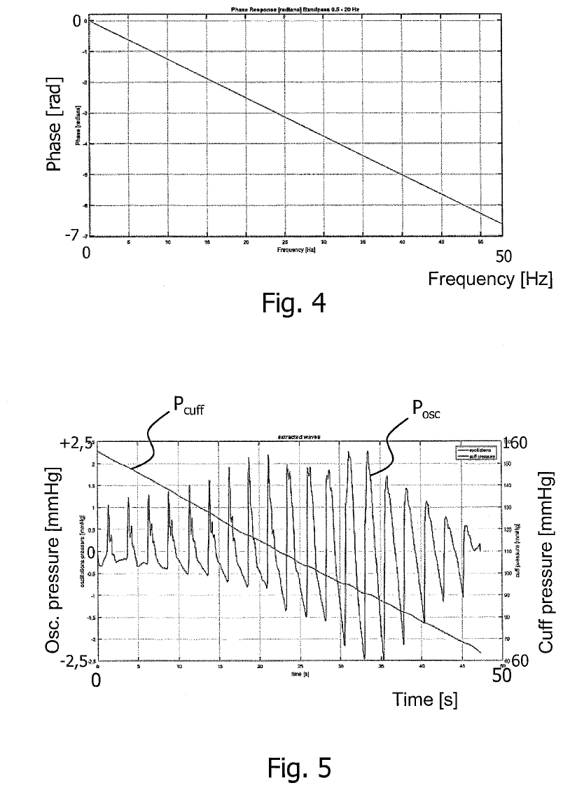

FIG. 4 shows an exemplary representation of the phase spectrum of the impulse response for an exemplary bandpass filter,

FIG. 5 shows an exemplary representation of a pressure signal broken down into oscillating and non-oscillating components,

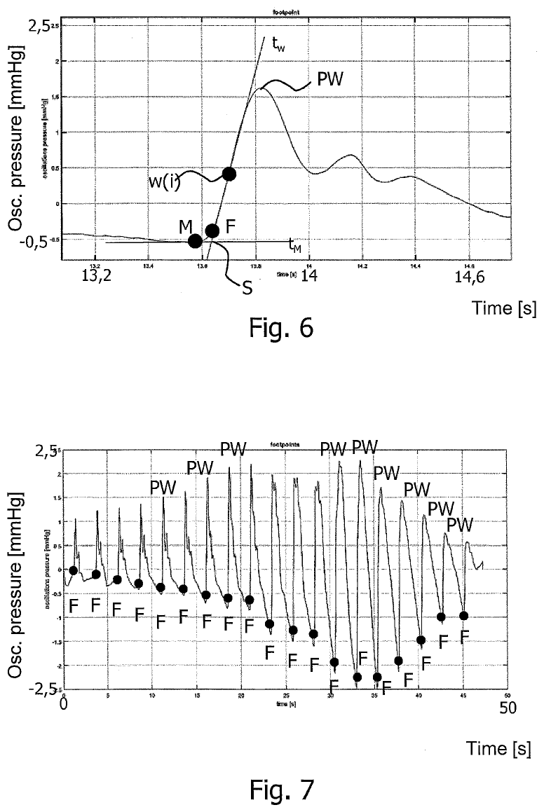

FIG. 6 shows an exemplary representation of a basepoint determination on a single pulse wave,

FIG. 7 shows an exemplary pulse wave sequence with successful basepoint detection,

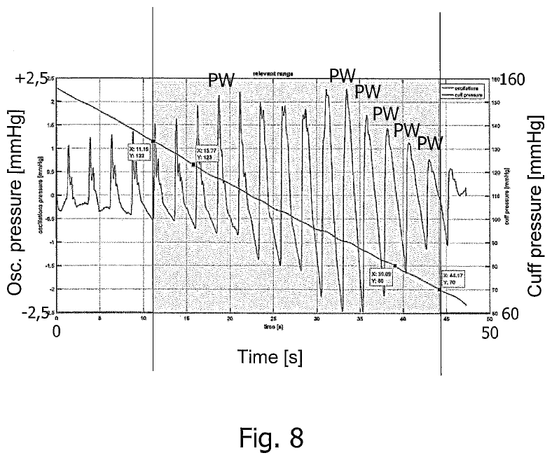

FIG. 8 shows an exemplary representation of the relevant pulse wave sequence range.

FIG. 1 shows a basic configuration for an arrangement for carrying out the method. The arrangement includes a cuff 1, which can be inflated by a pump 2 via a hose 2a. The cuff is applied around an extremity, for example around an upper arm. Furthermore, a pressure sensor 3 is provided, with which the time course of the pressure in the cuff is registered. The pressure sensor can be located at any position in the area of the cuff or pump system.

In the configuration presented here, the pressure sensor and the pump are combined in one housing together with further storage and data processing elements and a control and storage unit 4. The control and storage unit controls both the operation of the pump and the measured value acquisition by the pressure sensor. Especially under the control of the control and processing unit, the pump realizes e.g. a temporally linear pressure increase in the cuff during an inflation phase. During a release phase, an outlet valve not shown here, for example, causes the pressure in the cuff to drop linearly over time. These linear pressure curves are basically not necessary, but they prove to be useful and advantageous with regard to later artefact detection.

The control and storage unit 4 contains a control and evaluation program 5, which carries out the evaluation steps described in more detail below. In this example, the control and evaluation program is stored on an external storage medium 6 such as an SD card, a USB stick or another storage medium. It can be loaded into the control and storage unit 4 via an interface 7, for example an SD slot or a USB connection. Of course, it is also possible to permanently store the control and evaluation program in the control and storage unit in the form of a fixed firmware or firmware that can be updated via the interfaces.

The control and storage unit 4 contains an additional interface 8 for reading out the stored measurement data and for transferring the data to an external evaluation unit 9, for example a PC. The external evaluation unit has an evaluation program 10 with which the stored read-out data can also be analyzed. Interface 8 can be either wireless or wired. It is also possible to transfer data via a communication network to a remote host, so that remote monitoring and evaluation of blood pressure parameters is possible.

In the present case, the control and storage unit 4 also contains a display 11 for direct output of the measured blood pressure data and the temporal pressure curve in the cuff 1. Further operating elements may be provided, in particular for switching between a conventional blood pressure measurement and an operating mode in which the method according to the invention is carried out.

As mentioned above, one of the essential aspects of the method according to the invention is that the blood pressure signals during the measurement when inflating and/or deflating the cuff are both recorded and evaluated in their temporal course and also transformed for their later evaluation. As described, the apparatus consists of a cuff, a sensor and a pump. The pump inflates the cuff to a certain pressure. At the same time, the pulse waves during the process of inflating and/or deflating a cuff are recorded by monitoring the pressure curve within the cuff over time.

With the aid of special method steps described in more detail below, the pulse waves required for pulse wave analysis are then extracted from the pressure curve recorded over time during the inflation phase and/or the release phase on the arm or another limb. After subsequent processing, the signals of the extracted pulse waves are transformed to values that provide more accurate information about the aortal pulse waves. Interferences within the signal and artefacts of the measurements are detected and excluded from the analysis and/or corrected or signaled accordingly.

FIG. 2 shows an exemplary temporal signal curve of the recorded pressure in the cuff according to the procedure provided for this purpose. It can be seen that the pressure first increases in an initial inflation phase Inf and then decreases again in a subsequent release phase Def.

It can be seen that the pressure increases essentially linearly in the inflation phase and decreases essentially linearly in the release phase. Both the course of the linear pressure increase and the course of the linear pressure decrease are overlaid by an oscillatory signal component, which is shown here in the form of jagged lines in the pressure curve. This oscillatory signal component contains the information about the aortal pulse wave. According to the invention, it is extracted from the temporal pressure curve, wherein the exemplary method steps explained below are applied.

In order to record the temporal pressure curve shown in FIG. 2, a conventional blood pressure measurement is first carried out. In contrast to conventional blood pressure measurement, in which, for example, only the maximum amplitude of the oscillating signal component is registered, the method according to the invention records the temporal pressure curve Pₘₑₐₛ as a whole. This data acquisition can be done digitally in particular. The sampling rate and resolution is device-dependent. For example, a sampling rate of 40 Hz at a resolution of 12 bits is used.

Both the sampling rate and the resolution are not fixed per se, but only device-specific quantities. In order to guarantee platform independence and thus the best possible processability, the measured value quantity of the temporal pressure curve is then transformed to a uniform standard either in the control and storage unit itself or in the external evaluation unit so that its processing is platform-independent and thus uniform. For this purpose, the signal of the temporal pressure curve can be sampled up to a sampling frequency of 100 Hz, for example. Various approximation methods can be used for this sampling process. In particular, the so-called cubic spline approximation is used for this purpose. This approximation approximates two adjacent sample points.

For the cubic spline approximation, polynomials of degree 3 are determined for two adjacent sample points (xᵢ,yᵢ) and (xᵢ+1,yᵢ+1):

sᵢ(x)=aᵢ+bᵢ(x−xᵢ)+cᵢ(x−xᵢ)²+dᵢ(x−xᵢ)³

Because of the continuity of the signal to be sampled, the continuity requirement sᵢ(xᵢ+1)=sᵢ+1(xᵢ+1) applies, wherein s must be continuously differentiable twice:

sᵢ′(xᵢ₊₁)=sᵢ₊₁′(xᵢ₊₁)

sᵢ″(xᵢ₊₁)=sᵢ₊₁″(xᵢ₊₁)

When a new Δx selected, the signal can be resampled.

If the output signal is upsampled to a sampling frequency of 100 Hz, the following applies in this case

$$Δ x = \frac{1}{100}$$

However, the choice of the type of spline with regard to the edges does not play a decisive role.

From this platform-independent sampled signal, the pulse waves, i.e. the oscillatory component P_{osc} of the measured temporal pressure curve Pₘₑₐₛ, are extracted in the next step. The extraction is carried out, for example, by using an IIR bandpass filter. An FIR filter can also be used. Possible here is a Butterworth 6th order filter.

FIGS. 3 and 4 show exemplary filter characteristics. FIG. 3 shows the filter magnitude stated in dB as a function of frequency in the range from 0 to 50 Hz. FIG. 4 shows an exemplary phase response of the bandpass filter in the frequency range from 0 to 50 Hz. The bandpass range covers a frequency range from 0.5 Hz to 20 Hz. It is already adapted to the expected frequencies of the pulsating signal component. FIG. 3 shows that the magnitude of the filter characteristic curve in this range extends quasi horizontal and only decreases at higher frequencies of more than 20 Hz. The filter has the property of a zero-phase filter to avoid falsification of the phase spectrum of the processed signal.

An additive relationship can be applied to the measured temporal pressure signal. The measured pressure curve over time is the sum of the cuff pressure generated by the pump and the pulse pressure generated by the pulse wave. It therefore applies:

Pₘₑₐₛ=P_{cuff}+P_{osc}

Pₘₑₐₛ is the temporal pressure curve measured by pressure sensor 3, P_{cuff} is the pump pressure generated by pump 2, i.e. the cuff pressure, and P_{osc} are the pressure oscillations generated by the pulse wave on the arm or another limb, from which the characteristics of the aortal pulse waves are ultimately to be determined.

The bandpass range to be used for the bandpass filter can be derived from the following considerations. Correspondingly different operating conditions naturally require a basic configuration that deviates therefrom. The measurement takes place on the test person, e.g. at rest and in a sufficiently defined physiological state. Under these conditions, clearly definable heart rates in the range of 40 to 100 beats per minute can be assumed. This results in correspondingly defined cut-off frequencies for the oscillatory component P_{osc} in the temporal pressure curve.

If Hr_Low is the low heart rate of 40 beats per minute and Hr_High is the high heart rate of 100 beats per minute, this results in a lower cut-off frequency f_{L} and an upper cut-off frequency f_{H} by f_{L}=Hr_Low/60=40/60≈0.7 Hz and f_{H}=Hr_High/60=100/60≈1.7 Hz respectively.

The energy of a pulse wave generated by the beating heart is distributed over a certain frequency spectrum. For example, at a heart rate of 60 beats per minute, the largest portion of the energy of the pulse wave is in the range of 1-10 Hz, distributed over 10 harmonic oscillations.

Thus, if a range of 10 harmonic oscillations is assumed and the upper limits are taken into account, a frequency range of 0.7 Hz to 10-1.7 Hz, i.e. from 0.7 to 17 Hz, results for the pulse wave. However, these filter limits are not sharply defined. An additional interval is therefore added in each case on both sides of the frequency interval, so that a range of 0.5-20 Hz is selected as the frequency interval for the bandpass filter.

The oscillatory component P_{osc} of the measured temporal pressure signal Pₘₑₐₛ, and thus the pulse wave extracted from the measured signal, thus results from applying the filter function of the bandpass filter to the measured pressure signal:

P_{osc}=filter(Pₘₑₐₛ)

If the pulse wave is extracted by the filter, it follows from the above additive relationship that the remaining part of the originally recorded pressure signal is thus the cuff pressure generated by the pump.

P_{cuff}=Pₘₑₐₛ−P_{osc}

The measured pressure signal Pₘₑₐₛ is thus broken down into the cuff pressure P_{cuff} and the pulse oscillations P_{osc}.

FIG. 5 shows an example of the oscillatory component P_{osc} obtained from the output signal. The cuff pressure P_{cuff} can then ideally show a non-oscillating, almost linear rise in the area of the inflation phase and a non-oscillating and at least almost linear fall in the area of the release phase, provided that the pump generates a pressure increase in the cuff that is linear over time and provided that a pressure drop is generated that is linear over time when the pressure is deflated. FIG. 5 shows that these requirements are sufficiently well met in the example presented here.

If the characteristics of the inflation or deflation, i.e. the time characteristic of the cuff pressure during inflation realized by the pump or the time characteristic of the cuff pressure during deflation realized by the deflation valve, are known in advance, these characteristics can be checked for accuracy using the separated cuff pressure function P_{cuff}. This offers the possibility to calibrate the measuring arrangement or to determine additional artefacts in the measuring process.

If, for example, the linear characteristic of the linear drop in cuff pressure is known in advance, the actually measured and separated cuff pressure P_{cuff} can be examined for linearity. Strong deviations of the cuff pressure P_{cuff} from the linearity can then be interpreted as motion artefacts or other falsifying influences on the measuring process. This makes it possible to obtain an error signal which can be output to the measuring system to signalize the error. Accordingly, other non-linear characteristics can also be used to evaluate artefacts or a remaining residual.

Possible methods for the determination of the residual and a comparative test between the previously known characteristic curve and the time course of the measured cuff pressure P_{cuff} are distance determinations of the values of the cuff pressure P_{cuff} for the regression of the function to a known polynomial of n^{th} degree. In a test for linearity, the polynomial is accordingly a polynomial of degree 1.

The cuff pressure P_{cuff} is thereby subjected to a regression in one step. In a second step, the resulting regression relationship regression(P_{cuff}) is then compared with an n^{th} degree polynomial. The parameter of the artefact is then determined by the following relationship:

artefact=|regression(P_{cuff})−pⁿ|>threshold

with pⁿ as a comparison polynomial of n^{th} degree. This means that a measurement artefact is present precisely when the amount of the difference between the regression relationship of the cuff pressure P_{cuff} to the comparison polynomial pⁿ exceeds a certain predefined threshold value. In such a case, the control and storage unit will output an error signal, for example. The remaining residual can also provide further valuable information about the investigation and quality of the signals.

Following the separation of the pulse-like signal component P_{osc} from the cuff pressure P_{cuff}, the pulse wave signal is evaluated as such. The sub-steps carried out are first the decomposition of the pulse-like signal component into individual periods and a subsequent analysis of each individual, but at least one individual period itself, because the data contained here represent the properties of the individual pulse wave to be determined.

The identification of each individual pulse wave in the pulse-like signal component is carried out by means of a time derivative of the signal, for example by the control and storage unit with the aid of the control and evaluation program contained in this unit. The execution of the first time derivative with a subsequent basepoint determination has proven to be suitable, but is not limited to it.

With the basepoint method described here as an example, all turning points are determined with a positive slope. These are characterized in the first derivative precisely by the fact that the value of the first derivative is extreme in its place:

$$w = arg max \frac{{dP}_{osc}}{dt}$$

w are the respective positions of the “positive” turning points. The result is a set of all determined deflections, i.e. peaks, within the pulse-like signal component.

Then all peaks of the 1^{st} derivative are sorted in descending order of size.

$$s = sort ( \frac{{dP}_{osc}}{dt} ( w ) )$$

where s is the sorted sequence of the determined turning points.

This sorted sequence s of peaks is analyzed step by step. Each peak is recorded as a turning point if its minimum height is greater than 0 and if its distance to all turning points already recorded is greater than a defined time interval with respect to a given sample rate FS.

Each peak w(i) from the sequence s(i) is therefore a turning point idx if the following conditions are simultaneously fulfilled:

(a)s(i)>0 and

$$( b ) idx = \{ w ( i ) \vert s ( i ) > 0 ⋀ \vert \frac{w ( i ) - idx ( j )}{FS} \vert \cdot 1000 > D , mit j < i \}$$

Condition (b) expresses that a peak w(i) must have a certain minimum distance D to an already identified peak idx(j). This minimum distance is

(a)s(i)>0 and

$$( b ) idx = \{ w ( i ) \vert s ( i ) > 0 ⋀ \vert \frac{w ( i ) - idx ( j )}{FS} • 1000 > 2 ? , Tii . it / < i \text{?} \\ \text{?} \text{indicates text missing or illegible when filed}$$

independent of the sample rate. Here, for example, this is 350 ms.

A subsequent ascending sorting of idx puts the indexes in the correct chronological order.

In order to avoid possible noise and small disturbances in the signal and even more so in differentiation, the 1^{st} derivative can be calculated according to the “Savitzsky-Goley” method if necessary.

The position of their respective basepoints is of decisive importance for the identification of the individual pulse wave in the pulsating signal component. These can be determined from the turning points.

Starting from the respective determined turning point, the basepoints of the individual pulse waves are localized, for example, with the help of intersecting tangents in the vicinity of each individual maximum in the 1^{st} derivative of the P_{osc} signal, i.e. in the vicinity of the previously determined turning point. FIG. 6 shows an illustration of this. Besides the determined tangent t, at the turning point w(i), the first local minimum M in the time range before the pulse wave is searched for. This local minimum M has a horizontal tangent t_{M}. Then the intersection point S between the horizontal tangent t_{M} and the regression line, i.e. the tangent t_{w, is} calculated. This intersection point S, projected onto the signal, is the basepoint F of the pulse wave PW. This point can be adjusted additionally.

For each basepoint F with the coordinates Fⱼ(x, y) the following applies

xᵢ=idx(f)−i·gap

wherein gap means the respective gap in the samples, and yᵢ=P_{osc} (xᵢ) with i=1, . . . , n the regression points next to the turning point idx(j), wherein n is the number of regression points. Based on the coordinates (xᵢ, yᵢ) of the basepoints Fⱼ, coefficients a,b of a straight line equation g(x)=ax+b can now be determined. FIG. 7 illustrates a series of determined basepoints F in the pulse component of the signal, each of which includes individual pulse waves between them, some of which are marked with the reference sign PW as an example.

In a next step, a relevant part of the oscillatory signal component P_{osc} is defined with the determined individual pulse waves PW. FIG. 8 shows an illustrative example diagram. For this purpose, a meaningful pressure value is assumed, which is related to the systolic and diastolic blood pressure value. The pressure range of the pulse signal lies in particular between a pressure value of, for example, 10 mmHg above the systolic value and 10 mmHg below the diastolic pressure value.

A heart period is then defined as a section of two consecutive basepoints Fᵢ determined as before. The pulse pressure PP of each period is determined from the difference between systole and diastole.

The average pressure can be determined by the following relationship:

$${y}_{m} = \frac{1}{T} {\int }_{0}^{T} ydt ≅ \frac{1}{N} {\sum }_{i = 1}^{N} y ( i )$$

wherein T is the length of the time interval of period m over which averaging takes place, N is the number of samples over which averaging takes place, wherein y(i) is the amplitude of each individual registered sample i.

Subsequently, the mean pressure is subtracted from the pulse pressures of the period. This allows a so-called zero mean signal to be obtained.

Then the amplitudes of the individual pulse waves are scaled to the height of the measured pulse pressure PP.

However, not only the position and height of each individual pulse wave is of interest, but also its shape. This can be analyzed as follows:

The pulse waves in the inflation and/or release process have a different shape depending on the current cuff pressure. These forms can be classified into classes. Each class is represented by a predetermined calculation function, which reflects certain function courses of the pulse wave. Any number of calculation functions can be defined, whose values can be predefined as fixed variables in the control and storage unit, but also in the respective external evaluation devices. The number and design of the respective properties of the calculation functions can be changed flexibly.

The number of calculation functions, i.e. the number of classes, is k, wherein k is a natural number greater than 0.

Each pulse wave can then be assigned to such a class by the signal processing in the control and storage unit. Decisive for these classes are the different manifestations in the course of the individual pulse wave PW, respectively the existence of points and features of a first and a second shoulder of the individual pulse peak as well as a strength of a dicrotism or incision of the respective peak. Here, the first and second shoulder describes the strength of the “double peakness” of a peak, the dicrotism describes the change of the pulse wave due to the arrival of the reflected wave, while the incision indicates the expression of the dicrotism to a local minimum. Dicrotism and incision thus describe how “round” the individual period, i.e. the individual pulse wave, is in its course. Based on this, each individual pulse wave can be classified into shape classes.

The assignment to a class can be made according to the principle of the “nearest neighbor”. For example, the single pulse wave PW may have a left-side shoulder, whereas a first calculation function of class k=1 has no left-side shoulder and a second calculation function of class k=2 has a left-side shoulder. The single pulse wave thus deviates more from the shoulderless calculation function in its form than from the calculation function with the left-side shoulder. Thus the pulse wave PW belongs to class k=2 and is thus represented by the calculation function of class k=2.

Another possibility to assign a class is to perform the similarity analysis using a dynamic time equalization to make the period lengths and times invariant and allow a comparison. This comparison makes the shapes of the pulse waves comparable with each other and with the calculation functions and allows them to be assigned to classes.

For each of these classes, a class-specific algorithm can be specified, which determines the hemodynamic parameters for the class. A transfer function, consisting of a set of two subfunctions each, has proven to be suitable, but is not limited to it. These subfunctions describe the amplification of the amplitude spectrum and the shift of the phase spectrum. To determine the transformed signal, the amplitude and phase spectrum is determined from the signal. For this purpose, a Fast Fourier Transformation (FFT) is applied, for example.

The amplitude spectrum obtained therefrom is multiplied by the gain of the transfer function. The phase spectrum is added with the phase shift. The retransformation yields the aortic pulse curve of this specific class. Finally, the transformed data is evaluated to determine hemodynamic parameters.

To avoid discontinuities during transformation, the intermediate areas between classes are kept smooth. This means that if a pulse wave can be assigned to two classes, for example, each of these two classes is treated as equally probable.

If another pulse wave PW is now a little closer to one of these classes, the assignment is clear. For a coefficient \tilde{c} the following applies:

\tilde{c}=αcᵢ+(1+α)cᵢ₊₁,α=[0,1]

Cᵢ and Cᵢ₊₁ are the coefficients of the two closest neighbors.

The smoothness ensures that the two periods after transformation are not completely different, regardless of the class to which the first period was assigned.

After transformation, for each scaled pulse wave determined on the arm or another limb, an aortal pulse wave with its specific hemodynamic parameters is present. This information shall be weighted accordingly. It has proven to be suitable to determine certain parameters \tilde{x} together with a corresponding confidence interval \tilde{x} at the individual aortal pulse waves, but it is not limited to this. The confidence interval \tilde{x} to x is calculated as follows:

$$\tilde{x} = \overline{x} \pm 1 , 96 \frac{σ}{\sqrt{N}}$$

Here the parameter σ is the standard deviation and N is the number of periods. With increasing N the confidence interval decreases, a slow inflation and deflation rate, as well as a comparatively fast heart rate increase the accuracy. At low heart rates, the speeds of the inflation phase and the release phase should therefore be adjusted accordingly.

The width of the 95% confidence interval provides information about the reliability and quality of the parameter. Based on the extracted pulse waves determined on the arm or another limb and the calculated aortic pulse waves, it is possible to determine further hemodynamic parameters, such as aortic (central) blood pressure, for a comprehensive pulse wave analysis.

The method and according to the invention and the arrangement according to the invention were explained in more detail by means of embodiment examples. Further embodiments and designs result from the subclaims and within the scope of action by the person skilled in the art.

### LIST OF REFERENCE NUMERALS

1 Cuff
2 Pump
2a Hose
3 Pressure sensor
4 Control and storage unit
5 Control and evaluation program
6 External storage medium
7 Interface
8 Additional interface
9 External evaluation unit
10 External evaluation program
11 Display
Inf Inflation phase
Def Release phase
Pₘₑₐₛ Measured temporal pressure curve
P_{cuff} Cuff pressure
P_{osc} Oscillatory component of the pressure
F Basepoint
t_{w} Turning point tangent
t_{M} Tangent in minimum

1. Method for operating a blood pressure measuring device, comprising the following method steps:
performing a blood pressure measurement with the blood pressure measuring device, consisting of a cuff (1) for application around an extremity, a pump (2) for inflating the cuff and a pressure sensor (3) for registering the pressure within the cuff, wherein inflation of the cuff takes place in an inflation phase (Inf) and deflation of the pressure in the cuff in a release phase (Def),
registering and storing the temporal pressure curve (Pₘₑₐₛ) in the cuff (1) during the inflation phase and/or during the release phase in a control and storage unit (4), extracting a pulse-like signal component (P_{osc}) from the temporal pressure curve of the measured cuff pressure during the inflation phase and/or the release phase by means of a signal analysis within the control and storage unit (4),
signal analysis of the extracted pulse-like signal component (P_{osc}) within the control and storage unit with the steps of:
decomposition of the pulse-like signal component into data over individual periods to identify individual pulse waves (PW),
combining the data of the respective individual pulse waves (PW) from values of at least one calculation function from a predetermined calculation function set to generate a respective classified individual pulse wave (PW),
transforming the respective classified single pulse wave,
evaluating the transformed data to determine hemodynamic parameters.

2. Method according to claim 1,
characterized in that
the decomposition of the pulse-like signal component into data about the individual pulse waves (PW) is carried out in the time course of the pulse-like signal component (P_{osc}).

3. Method according to claim 1,
characterized in that
in combining the data of the respective individual pulse waves (PW) to generate the respective classified individual pulse wave, at least two calculation functions are combined with each other in a weighted manner.

4. Method according to claim 1,
characterized in that
the transformation of the respective classified single pulse wave into the period-specific amplitude and phase spectrum is carried out by means of a Fourier analysis unit.

5. Method according to claim 1,
characterized in that
a retransformation of the period-specific amplitude and phase spectrum of the classified individual pulse wave is performed to determine the time course of the aortal pulse wave.

6. Method according to claim 1,
characterized in that
the registration of the temporal pressure curve in the inflation phase and/or in the release phase is carried out digitally at a fixed device-specific sampling rate and a fixed device-specific resolution, wherein subsequent sampling to a platform-independent sampling rate is carried out in conjunction with an approximation of the measured temporal pressure curve (Pₘₑₐₛ) in the cuff.

7. Method according to claim 1,
characterized in that
the pulse-like signal component (P_{osc}) is extracted via a bandpass filter to separate a non-oscillating cuff pressure (P_{cuff}).

8. Method according to claim 1,
characterized in that
the separated non-oscillating cuff pressure (P_{cuff}) is checked for compliance with a standard curve, wherein a determination of an artefact parameter for indicating a measured value-distorting influence is carried out from the degree of deviation of the non-oscillating pump trend from the standard curve.

9. Arrangement for measuring the pressure in a blood vessel, comprising a cuff (1) for application around an extremity, a pump (2) for inflating the cuff, a pressure sensor (3) for registering the temporal pressure curve (Pₘₑₐₛ) applied in the cuff, and a control and storage unit (4) for operating the pump and the pressure sensor and for storing the temporal pressure curve (Pₘₑₐₛ),
wherein
the control and storage unit (4) has a control and evaluation program (5) for separating pulse-like components (P_{osc}) in the temporal pressure curve (Pₘₑₐₛ) during an inflation and/or release phase of the temporal pressure curve and for a signal analysis of the separated pulse-like components (P_{osc}).

10. Arrangement according to claim 9,
characterized in that
the control and evaluation program (5) can be loaded and updated from an external storage means (6) via an interface (7) onto the control and storage unit (4).

11. Arrangement according to claim 9,
characterized in that
the control and storage unit (4) has an interface (8) for reading out stored data, in particular the measured temporal pressure curve (Pₘₑₐₛ), to an external evaluation unit (10) and/or a remote host.

12. Arrangement according to claim 9,
characterized in that
the control and storage unit (4) has a display (11) for outputting parameters obtained from the pulse-like components of the pressure curve, in particular for outputting a heartbeat frequency and/or hemodynamic parameters from the pulse wave analysis and/or a curve of an aortic pulse wave.

13. Arrangement according to claim 9,
characterized in that
the control and storage unit (4) has a switching facility between a first operating mode for carrying out a standard blood pressure measurement and a second operating mode for carrying out a measurement of the pulse-like components of the temporal pressure curve and for determining parameters of an aortal pulse wave.

14. Use of a method according to claim 1 for determining and analyzing an aortic pulse wave and for determining hemodynamic parameters of the pulse wave analysis, in particular an aortic blood pressure.

15. Use of an arrangement according to claim 9 for determining and analyzing an aortic pulse wave and for determining hemodynamic parameters of the pulse wave analysis, in particular an aortic blood pressure.
